(19) United States
(12) Patent Application Publication
(10) Pub. No.: US 2023/0047214 A1
(43) Pub. Date: Feb. 16, 2023

(54) METHODS FOR MICROWAVE SYNTHESIS OF DEGRADABLE POLYMERS FOR DRUG DELIVERY

(71) Applicant: The General Hospital Corporation, Boston, MA (US)

(72) Inventors: Orhun K. Muratoglu, Cambridge, MA (US); Ebru Oral, Newton, MA (US); Scott Grindy, Boston, MA (US)

(21) Appl. No.: 17/796,925

(22) PCT Filed: Feb. 3, 2021
(86) PCT No.: PCT/US2021/016475

§ 371 Date: Aug. 2, 2022

Related U.S. Application Data
(60) Provisional application No. 62/969,247, filed on Feb. 3, 2020.

(51) Int. Cl. A61L 27/34; A61L 27/52; A61L 2/12; A61L 27/54; A61L 27/58; A61L 29/08; A61L 29/14; A61L 29/16; A61L 31/10; A61L 31/14; A61L 31/16; C08F 283/06

(52) U.S. Cl. CPC A61L 27/34; A61L 27/52; A61L 2/12; A61L 27/54; A61L 27/58; A61L 29/085; A61L 29/145; A61L 29/148; A61L 29/16; A61L 31/10; A61L 31/145; A61L 31/148; A61L 31/16; C08F 283/06; A61L 2300/402

(57) ABSTRACT

Provided herein are methods of making degradable, additive-blended polymeric materials using microwave radiation and catalysts. The methods can include incorporation of therapeutic materials into the polymeric materials. There also are provided polymeric materials made by the methods and medical devices comprising the polymeric materials made by the methods.

### STATEMENT REGRADING FEDERALLY FUNDED RESEARCH

This work was funded by the Office of the Assistant Secretary of Defense for Health Affairs, through the Peer Reviewed Medical Research Program under Award No. W81XWH-17-1-0614.

### FIELD OF THE INVENTION

The present invention relates to methods for synthesizing degradable polymeric materials using a microwave apparatus. It also relates to methods of incorporating therapeutic agents in the polymeric materials. It also relates to making medical devices comprised of polymeric materials incorporated with therapeutic agents.

### BACKGROUND OF THE INVENTION

Medical device associated infections are common complications that cause significant mortality and morbidity in patients. In the United States, it is estimated that annually 150,000 to 400,000 patients with bladder catheter or central venous catheter are infected respectively (Darouiche, R O. Device-Associated Infections: A Macroproblem that Starts with Microadherence. Clinical Infectious Diseases, 2001, 33:1567-1572). In the orthopedic field, 100,000 to 200,000 patients with fracture fixation devices, and 6,000 to 18,000 patients with joint prostheses are infected in U.S. annually. In the cardiovascular field, 5,000 to 23,000 patients with vascular grafts. 3,000 to 21,000 patients with cardiac pacemakers in US are infected annually. The actual rates of medical device associated infection might actually even be higher due to a variety of reasons, including increased rates of infection with re-implanted devices.

Medical devices have an increased susceptibility to bacterial colonization because of several factors. One reason is that; after implantation, the host immune system creates an immune-incompetent fibroinflammatory zone around the medical implant (Gristina A G. Implant failure and the immune-incompetent fibro-inflammatory zone. Clin Orthop Relat Res. 1994; 298:106-118). In addition, the biomaterial(s) used in manufacturing the medical device can induce adhesion of bacteria to the medical device. Adhesion of bacteria to the surface(s) of a medical device can then induce formation of bacterial biofilms that are much less accessible to antibiotics (Gristina A G. Implant failure and the immune-incompetent fibro-inflammatory zone. Clin Orthop Relat Res. 1994; 298:106-118).

Current treatment for medical device associated infections, e.g. prosthetic joints, pacemakers, fracture plates, intramedullary nails include removal of the device, debridement of potentially infected tissues surrounding the implants, re-implantation with new implants, and administration of systemic antibiotics. Alone, parenteral administration of antibiotics is not effective for treating medical device associated infections because the antibiotic penetration to the site of infection depends on the blood flow to the infected site. Areas with relatively low blood flow (e.g. bone, cartilage, immediate area surrounding medical implants) will have low local concentration of antibiotics, while areas with relatively high blood flow (e.g. liver, kidney) will have high concentration of antibiotics. For example, only 7-15% of intravenous cefazolin, 5-20% of intravenous vancomycin, and 5-19% of intravenous ceftriaxone penetrate bone (Spellberg B, Lipsky B A. Systemic Antibiotic Therapy for Chronic Osteomyelitis in Adults. Clin Infect Dis. 2012, 54(3):393-407). For infections associated with joint replacements, in addition to removal of the device and debridement of potentially infected tissues surrounding the implants, a spacer shaped like the articulating joint made of antibiotic-loaded bone cement is placed into the joint. In addition, systemic antibiotics are administered for at least 6 weeks (while the patient is immobilized), and a second surgery is performed for reimplantation with new implants.

Several drug eluting polymers, such as non-degradable bone cements (PMMA-based in situ curing polymers) are available and in clinical use for the local delivery of antibiotics. For example, gentamicin containing bone cement that contains 1.0 gram gentamicin in a 40 g bone cement is commercially available under the trade name SmartSet® GHV Gentamicin, DePuy® CMW 2 Gentamicin, and Zimmer® Palacos R+G. Gentamicin containing bone cements can be used in total joint replacement both as prophylaxis or as treatment (Bourne R B. Prophylactic use of antibiotic bone cement an emerging standard-in the affirmative. J of Arthroplasty, 2004, 19(4), Suppl 1, 69-72). The gentamicin released from these bone cements reaches a maximum release rate of 10 μg/cm²/h shortly after implantation but decreases significantly to 0.1-1 μg/cm²/h by 24 hrs. and below 0.1 μg/cm²/hr by 1 week (Van de Belt H, Neut D, μges D R A, Schenk W, van Horn J R, van der Mei H C, Busscher H J. Surface roughness, porosity and wettability of gentamicin-loaded bone cements and their antibiotic release. Biomaterials, 2000, 21:1981-1987). Because the amount of antibiotic eluted is very low after 24 hrs. the commercially available antibiotic cements are ineffective as a single mode of treatment for infection (Gogja J S, Meehan J P, Di Cesare, P, Jamali A A. Local Antibiotic Therapy in Osteomyelitis. Semin Plast Surg, 2009, 23(2):100-107).

Biodegradable antibacterial envelope TYRX™ is available for clinical use to reduce infection associated with pacemakers and implantable cardioverter defibrillators (http://www.tyrx.com/wcm/groups/mdtcom_sg/@mdt/@corp/documents/documents/uc201405268d-clinician-s-br.pdf). The biodegradable antibacterial envelope is composed of a bioabsorbable polacrylate polymer that elutes rifampin and minocycline. A clinical study of 1,129 patients showed 80% fewer major cardiac implantable electronic device infections as compared to the control cohort (0.44% vs 2.2%, p=0.0023) (http://www.tyrx.com/wcm/groups/mdtcom_sg/@mdt/@corp/documents/documents/uc201503789a_citadel_centurion.pdf).

Exparel™ is a sustained-release formulation of the local anesthetic bupivacaine, and is available for clinical use to treat post-operative pain. The manufacturer claims that Exparel releases bupivacaine over approximately 72 hours after injection to provide pain relief. Exparel is composed of DepoFoam™, lipid-based particles which encapsulate the bupivacaine and release it over an extended period of time.

A major drawback of most current commercially available drug eluting polymers are that they are pre-manufactured with the drug, and therefore medical professionals are unable to select the most specific drug for the patient and use the polymer to create a custom drug eluting polymer. Flexibility in using the desired drug or combination of drugs is desirable in applications such as when using antibiotics to treat peri-prosthetic implant infections. For effective doses of treatment drugs to be delivered, flexibility in manipulating drug elution rates and allowing effective dose delivery over desired periods of time is desirable. Biodegradability is desired because after all the drug has been eluted from a drug-eluting polymer, it could be a potential surface for colonization by bacteria and therefore susceptible to recurrent medical device infection.

### BRIEF DESCRIPTION OF THE DRAWINGS

FIG. 1: The chemical structure of the liquid macromers.

FIG. 2: Cross-sectional optical microscope image showing the incorporation of Bupivacaine Hydrochloride into MA-PLA₄-PEG₉-PLA₄-MA solid gels.

FIG. 3: Cross-sectional optical microscope image showing the incorporation of Bupivacaine Hydrochloride into MA-PLA₄-PEG₁₄-PLA₄-MA solid gels.

FIG. 4: Total Bupivacaine released from solid gels over 2 weeks.

FIG. 5: A solid gel layer of MA-PLA4-PEG14-PLA4-MA polymerized on top of ultra-high molecular weight polyethylene containing 0.5% 4-hydroxybenzophenone.

FIG. 6: Fourier-transform infrared spectra of MA-PLA₂-PEG₃₄-PLA₂-MA macromer and a MA-PLA₂-PEG₃₄-PLA₂-MA solid gel.

FIG. 7: Proton nuclear magnetic resonance spectrum of PCL₄-PEG₂₃-PCL₄.

FIG. 8: Proton nuclear magnetic resonance spectrum of PLA₄-PEG₂₃-PLA₄.

FIG. 9: Proton nuclear magnetic resonance spectrum of MA-PLA₄-PEG₂₃-PLA₄-MA.

FIG. 10: Degradation of MA-PLA₄-PEG₁₄-PLA₄-MA.

FIG. 11: Ketorolac release from MA-PLA4-PEG4.5-PLA4-MA gel

### SUMMARY OF THE INVENTION

The present invention relates generally to methods of making implantable biological devices from degradable polymeric materials, preferably containing therapeutic agents. The therapeutic agents can be delivered at the site of the implantation. The delivery rate of the therapeutic agents can be made modified through changing the methods of making the devices. The present invention particularly describes novel and practical methods of synthesizing macromers, which can be mixed with desired therapeutic agent(s) and polymerized into degradable delivery devices. The present invention particularly described methods of synthesizing macromers using microwave radiation.

In some embodiments, the present invention describes methods of synthesizing macromers; which are block co-polymers, where one of the polymer blocks is degradable and one of the polymer blocks is cross-linkable. In some embodiments, the invention describes methods of making materials by polymerizing said macromers via stimulating their cross-linkable moieties. In some embodiments, the invention describes methods of making medical implants compromising polymers made by the polymerization of said macromers. In some embodiments, the invention describes methods of making medical implants integrated with polymers made by the polymerization of said macromers.

Preferably, the polymerizable macromers described in this invention are in liquid form at room temperature, thus may be referred to as ‘liquid polymerizable mixture’. The process comprises steps of synthesizing the block co-polymer macromer, blending the macromer with therapeutic agents, initiator molecules, and additives, and polymerizing the macromer to produce a solid gel. The invention also describes methods of making medical implants comprising gels, machining and sterilizing them.

This invention also describes methods to make devices where the solid gel formed from block co-polymer macromers is integrated as part of another device. The invention also describes methods to create interlocked hybrid materials with integrated solid gel formed from described macromers.

In one embodiment, the invention provides methods of making a degradable, additive-blended polymeric material comprising.
(a) Providing a first polymeric material for a central moiety (Aₙ),
(b) Contacting the first polymeric material with a monomer B,
(c) Initiating a reaction between the first polymeric material and the monomer by microwave radiation, thereby forming a block copolymer with blocks composed of the starting polymeric material and the polymer of the monomer (Bₘ-Aₙ-Bₘ),
(d) Optionally washing the block copolymer with a solvent or solvents,
(e) Contacting the block copolymer with a second monomer (R),
(f) Initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(g) Optionally washing the liquid polymerizable macromer with a solvent or solvents,
(h) Contacting one or more liquid, polymerizable macromer(s) with initiator(s),
(i) Blending the liquid, polymerizable mixture with additive(s),
(j) Exposing the additive-blended, liquid polymerizable macromer(s) to as external stimulus which can create free radicals for a period of time, thereby forming a degradable, additive-blended gel.

In any of the embodiments where a polymeric material Aₙ is provided as a central moiety, and B is provided as the first monomer and degradable moiety, the final macromers containing different polymerization degrees n and m can be mixed in any fraction before blending with additive(s) and polymerization into solid gel form. One or more initiators can be blended into the mixture before polymerization into solid gel form.

In one embodiment, the invention provides methods of making a degradable, additive-blended medical implant comprising:
(a) Providing a first polymeric material (Aₙ),
(b) Contacting the first polymeric material with a monomer B,
(c) Initiating a reaction between the first polymeric material and the monomer by microwave radiation, thereby forming a block copolymer with blocks composed of the starting polymeric material and the polymer of the monomer

(Bₘ-Aₙ-Bₘ),
(d) Optionally washing the block copolymer with a solvent or solvents,
(e) Contacting the block copolymer with a second monomer (R),
(f) Initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(g) Optionally washing the liquid polymerizable macromer with a solvent or solvents,
(h) Contacting the liquid, polymerizable macromer with initiator(s),
(i) Blending the liquid, polymerizable mixture with additive(s).
(j) Exposing the additive-blended, liquid polymerizable macromer(s) to as external stimulus which can create free radicals for a period of time, thereby forming a medical implant.

In any of the embodiments describing methods of making a medical implant, the steps of making a medical implant can be performed in a sterile environment or the medical implant can be terminally sterilized before implantation.

In one embodiment, the invention provides methods of making a sterile, degradable, additive-blended medical implant comprising:
(a) Providing a first polymeric material (Aₙ),
(b) Contacting the first polymeric material with a monomer B,
(c) Initiating a reaction between the first polymeric material and the monomer by microwave radiation, thereby forming a block copolymer with blocks composed of the starting polymeric material and the polymer of the monomer (Bₘ-Aₙ-Bₘ),
(d) Optionally washing the block copolymer with a solvent or solvents,
(e) Contacting the block copolymer with a second monomer (R),
(f) Initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(g) Optionally washing the liquid polymerizable macromer with a solvent or solvents,
(h) Contacting the liquid, polymerizable macromer with initiator(s),
(i) Blending the liquid, polymerizable mixture with additive(s),
(j) Exposing the additive-blended, liquid polymerizable macromer to as external stimulus which can create free radicals for a period of time, thereby forming a degradable, additive-blended gel;
(k) Shaping or machining the degradable, additive-blended gel into a medical implant;
(l) Packaging and sterilizing the medical implant.

In one embodiment, the invention provides methods of making a degradable, additive-blended polymeric material comprising:
(a) Providing a first polymeric material (Aₙ),
(b) Contacting the first polymeric material with a monomer B,
(c) Initiating a reaction between the first polymeric material and the monomer by microwave radiation, thereby forming a block copolymer with blocks composed of the starting polymeric material and the polymer of the monomer (Bₘ-Aₙ-Bₘ),
(d) Optionally washing the block copolymer with a solvent or solvents,
(e) Contacting the block copolymer with a second monomer (R),
(f) Initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(g) Optionally washing the liquid polymerizable macromer with a solvent or solvents,
(h) Contacting the liquid, polymerizable macromer with initiator(s),
(i) Blending the liquid, polymerizable mixture with therapeutic agent(s),
(j) Exposing the additive-blended, liquid polymerizable macromer to as external stimulus which can create free radicals for a period of time, thereby forming a degradable, additive-blended gel.

In one embodiment, the invention provides methods of making an antibacterial, degradable, additive-blended polymeric material comprising:
(a) Providing a first polymeric material (Aₙ),
(b) Contacting the first polymeric material with a monomer B,
(c) Initiating a reaction between the first polymeric material and the monomer by microwave radiation, thereby forming a block copolymer with blocks composed of the starting polymeric material and the polymer of the monomer (Bₘ-Aₙ-Bₘ),
(d) Optionally washing the block copolymer with a solvent or solvents,
(e) Contacting the block copolymer with a second monomer (R),
(f) Initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(g) Optionally washing the liquid polymerizable macromer with a solvent or solvents,
(h) Contacting the liquid, polymerizable macromer with initiator(s),
(i) Blending the liquid, polymerizable mixture with antibacterial agent(s),
(j) Exposing the additive-blended, liquid polymerizable macromer to as external stimulus which can create free radicals for a period of time, thereby forming an antibacterial, degradable, additive-blended gel.

In one embodiment, the invention provides methods of making a therapeutic, degradable, additive-blended polymeric material comprising:
(a) Providing a first polymeric material (Aₙ),
(b) Contacting the first polymeric material with a monomer B,
(c) Initiating a reaction between the first polymeric material and the monomer by microwave radiation, thereby forming a block copolymer with blocks composed of the starting polymeric material and the polymer of the monomer (Bₘ-Aₙ-Bₘ),
(d) Optionally washing the block copolymer with a solvent or solvents,
(e) Contacting the block copolymer with a second monomer (R),
(f) Initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(g) Optionally washing the liquid polymerizable macromer with a solvent or solvents,
(h) Contacting the liquid, polymerizable macromer with initiator(s),
(i) Blending the liquid, polymerizable mixture with analgesic(s) and non-steroid inflammatory(ies),
(j) Exposing the additive-blended, liquid polymerizable macromer to as external stimulus which can create free radicals for a period of time, thereby forming an antibacterial, degradable, additive-blended gel.

In one embodiment, the invention provides methods of making an antibacterial, degradable, additive-blended integrated polymeric material comprising;
(a) Providing a first polymeric material (Aₙ),
(b) Contacting the first polymeric material with a monomer B,
(c) Initiating a reaction between the first polymeric material and the monomer by microwave radiation, thereby forming a block copolymer with blocks composed of the starting polymeric material and the polymer of the monomer (Bₘ-Aₙ-Bₘ),
(d) Optionally washing the block copolymer with a solvent or solvents,
(e) Contacting the block copolymer with a second monomer (R),
(f) Initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(g) Optionally washing the liquid polymerizable macromer with a solvent or solvents,
(h) Providing a second polymeric material;
(i) Contacting the liquid, polymerizable macromer with initiator(s),
(j) Blending the liquid, polymerizable mixture with analgesic(s) and non-steroid inflammatory(ies),
(k) Applying the additive-blended liquid, polymerizable macromer to the second surface;
(l) Exposing the additive-blended, liquid polymerizable macromer to as external stimulus which can create free radicals for a period of time, thereby forming an antibacterial, degradable, additive-blended integrated polymeric material.

In some embodiments, a second polymeric material is provided on which to apply the additive-blended liquid, polymerizable macromer. This second polymeric material can itself be previously blended with additives such as antioxidants, cross-linking agents, initiators, and/or therapeutic agents. This second polymeric material can previously have gone other treatments such as compression molding, pelletization, crosslinking, high temperature melting. This second polymeric material can be a polyethylene-based material such as ultrahigh molecular weight polyethylene, high density polyethylene, low density polyethylene and/or a mixture thereof.

In one embodiment, the invention provides methods of making an antibacterial, degradable, additive-blended integrated material comprising:
(a) Providing a first polymeric material (Aₙ),
(b) Contacting the first polymeric material with a monomer B,
(c) Initiating a reaction between the first polymeric material and the monomer by microwave radiation, thereby forming a block copolymer with blocks composed of the starting polymeric material and the polymer of the monomer (Bₘ-Aₙ-Bₘ),
(d) Optionally washing the block copolymer with a solvent or solvents,
(e) Contacting the block copolymer with a second monomer (R),
(f) Initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(g) Optionally washing the liquid polymerizable macromer with a solvent or solvents,
(h) Providing a second material;
(i) Contacting the liquid, polymerizable macromer with initiator(s),
(j) Blending the liquid, polymerizable mixture with analgesic(s) and non-steroid inflammatory(ies),
(k) Applying the additive-blended liquid, polymerizable macromer to the second material;
(l) Exposing the additive-blended, liquid polymerizable macromer to as external stimulus which can create free radicals for a period of time, thereby forming an antibacterial, degradable, additive-blended integrated material.

In some embodiments, a second material is provided on which to apply the additive-blended liquid, polymerizable macromer. This second material can be made of a metal or a ceramic in addition to a polymer. It can have a porous, solid or partially porous surface. This surface can be pretreated, for example to passivate or oxidize the surface.

### DETAILED DESCRIPTION

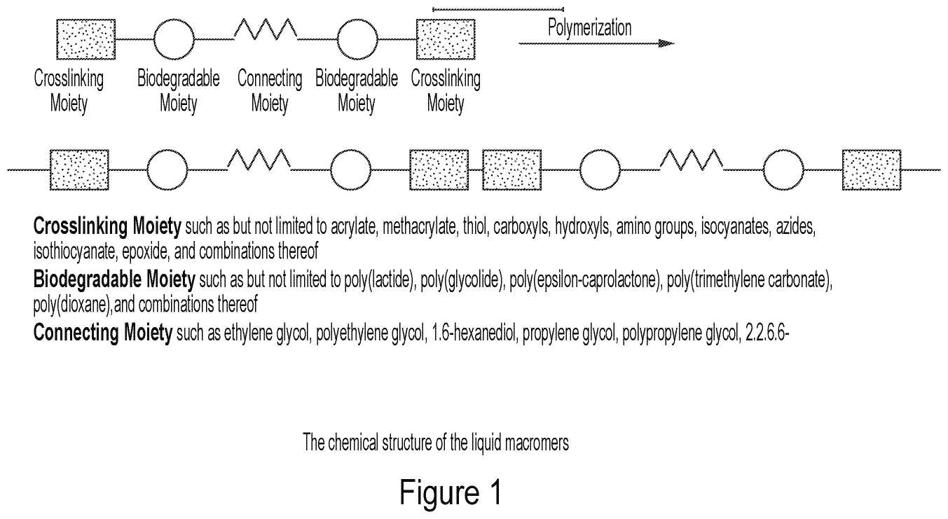

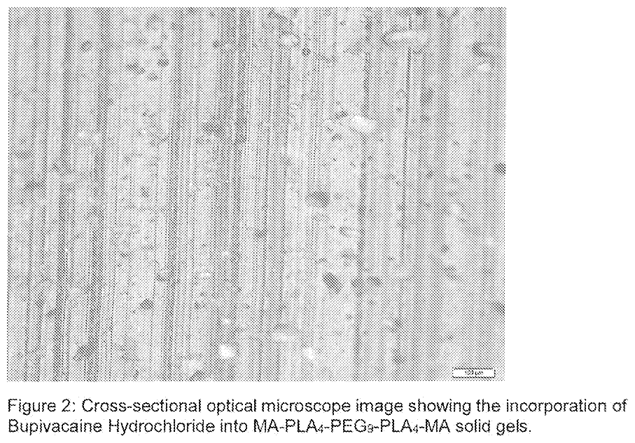

In this invention, the embodiments describe methods of synthesizing macromer(s), which can be mixed with other additives including therapeutic agents and polymerized into degradable therapeutic agent-loaded polymeric networks. The macromonomer or macromer mixture is liquid, biodegradable, and cross-linkable at ambient conditions, at room temperature or at elevated temperatures. What is meant by room temperature is between about 0° C. and about body temperature or about 40° C. The cross-linking can also take place at temperatures higher than 40° C., for example at about 50° C., or at about 60° C., or higher. The macromer(s) are composed of a connecting moiety and biodegradable moiety and a cross-linkable moiety. In one embodiment, the macromer is composed of two biodegradable moieties connected by a central/connecting moiety and end capped with two or more cross-linkable moieties (FIG. 1). In this invention, most often a central connecting moiety is mentioned, but connecting moieties can be placed in between cross-linkable and biodegradable moieties as well. In another embodiment, the macromer is composed of one or more biodegradable moiety(ies) and end capped with two or more cross-linkable moiety(ies).

Synthesis Reaction 1—Microwave Treatment

In a preferred embodiment, the chemical reaction between the polymeric starting material (central/connecting moiety) and the second monomer is initiated by microwave irradiation. The resultant of this reaction is the block copolymer consisting of the central moiety and the degradable moieties. In one embodiment, the polymeric starting material is mixed with monomer(s) and catalyst(s) and the mixture placed in a microwave oven and exposed to microwave radiation. The duration of microwave treatment can be anywhere between 1 second to several hours or more. In any of the embodiments, the power of the microwave used can be 50 W, 60 W, 70 W, 80 W, 90 W, 100 W, 200 W, 300 W, 400 W, 500 W, 600 W, 700 W, 800 W, 900 W, 1000 W, 1100 W, 1200 W or more than 1200 W or less than 50 W or any value in between. The microwave treatment can be carried under atmospheric pressure, under pressures lower than atmospheric pressure, under partial vacuum, with or without active heating, such radiant heating or convection heating. The duration of microwave treatment can be anywhere between 1 second to several hours or more.

Microwave treatment can also be performed on different volumes of mixtures of polymeric starting material and second monomer. In those cases, the duration of exposure to microwave radiation can be modified (made longer or shorter) to homogeneously heat the mixture and initiate the chemical reaction.

In some embodiments, the intensity of microwave irradiation may vary throughout the cavity which contains the microwave energy (‘microwave oven’). In such cases, the mixture of polymeric starting material and second monomer may be chosen in specific areas of intensity to increase or decrease the amount of microwave energy absorbed by the mixture of polymeric starting material and second monomer.

In some embodiments, the central/connecting moiety or polymeric starting material is polyethylene glycol (PEG; Aₙ) and its molecular weight molecular is about 150-100,000 g/mol but ideally 150-2000 g/mol. In some embodiments, the second monomer B is DL-lactide or L-lactide or D-lactide and its corresponding polymer segment (degradable moiety) is poly(D,L-lactic acid) (PDLLA) or poly(D-lactic acid) (PDLA) or poly(L-lactic acid) (PLLA) and the corresponding block co-polymer is (PDLLA-Aₙ-PDLLA) or (PDLA-Aₙ-PDLA) or (PLLA-Aₙ-PLLA), or the second monomer can be a mixture of these monomers. In some embodiments, the second monomer B is glycolide and its corresponding polymer segment is poly(glycolic acid) (PGA) and the corresponding block co-polymer is (PGA-Aₙ-PGA). In one embodiment, the second monomer B is a mixture of lactide and glycolide, and the corresponding random co-polymer is poly(lactic acid-co-glycolic acid) (PLA-r-PGA) and the corresponding block co-polymer is ((PLA-r-PGA)-Aₙ-(PLA-r-PGA)).

In some embodiments, the number of repeats of the resulting degradable moeity in Bₘ-Aₙ-Bₘ), i.e. ‘m’ is about 1-20 but ideally 1-10.

The catalyst molecule may be from any of the classes of ring-opening catalysts, including organic catalysts, enzymatic catalysts, or metal catalyst systems. An exemplary catalyst is tin 2-ethylhexanoate. The catalyst can be added to the mixture at concentrations from 0.0001 to 10 wt/wt %, preferably 0.1-3 wt/wt %, most preferably 2 wt/wt %. The catalyst can be dissolved in a solvent; the solvent can be chosen from any of the organic solvents such as acetone, acetonitrile, benzene, butyl acetate, carbon tetrachloride, chloroform, cyclehexane, 1,2 dichloroethane, dichloromethane, dimethylformamide, dimethyl sulfoxide, dioxane, ethanol, ethyl acetate, diethyl ether, heptane, hexane, methanol, methyl-t-butyl ether, 2-butanone, pentane, n-propanol, isopropanol, diisopropyl ether, tetrahydrofuran, toluene, trichloroethylene, water, xylene, or no solvent may be used. The amount of solvent in the mixture can be from 1 to 99.9 wt/wt %, preferably 0-5 wt/wt %.

In some embodiments, after the second monomer is reacted with the polymeric starting material to create the block co-polymer, the mixture may be purified by washing with a solvent to remove impurities. Common solvents can be chosen from but are not limited to hexane, diethyl ether, or alcohols such as methanol or ethanol. The washing can be done in multiple steps, each step can use pure solvents or a mixture of solvents. The ratio of block co-polymer to solvent may vary from 1 to 50 volume/volume %, preferably 5-40 volume/volume %, most preferably 10-20 volume/volume %. The temperature of washing may vary from 0 degrees Celsius to 50 degrees Celsius, most preferably 20-30 degrees Celsius. After washing, the solvent may be removed by reduced pressure, elevated temperatures, or a combination of reduced pressure and elevated temperatures. The pressure may range from 20 millibar to 0.5 bar, most preferably 2-5 bar.

After washing, the co-polymer may be stored at temperatures between 0 and 100 degrees Celsius, under atmospheric conditions or under inert gases such as nitrogen, argon, or any other composition of gases. Most preferably, the co-polymer may be stored at temperatures between 20-30 degrees Celsius and under atmospheric conditions.

In one embodiment, the invention provides methods of making a degradable, additive-blended polymeric material comprising:
(a) Providing a first polymeric material for a central moiety (Aₙ),
(b) Mixing the first polymeric material with a monomer B in the presence of catalyst(s),
(c) Exposing the mixture to microwave radiation for a period of time, thereby forming a block copolymer with blocks composed of the starting polymeric material and the polymer of the monomer (Bₘ-Aₙ-Bₘ),
(d) Mixing the block copolymer with a second monomer (R),
(e) Exposing the mixture of the block copolymer and the second monomer to microwave radiation for a period of time, thereby forming a liquid polymerizable macromer composed of a cross-linkable moiety ((Bₘ-Aₙ-Bₘ—R) or (R—Bₘ-Aₙ-Bₘ—R)),
(f) Contacting one or more liquid, polymerizable macromer(s) with initiator(s),
(g) Blending the liquid, polymerizable mixture with additive(s),
(h) Exposing the additive-blended, liquid polymerizable macromer(s) to as external stimulus which can create free radicals for a period of time, thereby forming a degradable, additive-blended gel.

In some embodiments, the mixing of the reaction components of the first or second reactions can be done sequentially, or simultaneously. In any of the embodiments, multiple types of the components such as the central moiety or the monomers can be used. Multiple catalysts or solvents can be used.

In any embodiments, the microwave radiation exposure can be performed for a duration of at least 1 second up to several hours, more preferably about 15 seconds to several minutes, most preferable about 30 seconds to 3 minutes. In any of the embodiments, microwave radiation exposure can be performed in a precooled or preheated environment. It can also be performed with active heating or cooling. The heating or cooling can be done at any rate.

Synthesis Reaction 2—Microwave Treatment

In a preferred embodiment, the chemical reaction between the reaction product of the first synthesis reaction, that is the block copolymer of the central/connecting moiety and the degradable moieties, and the second monomer is initiated by microwave irradiation. The resultant of this reaction is the macromer consisting of the central moiety, degradable moieties and cross-linkable moieties (FIG. 1). The second monomer can be a precursor that can react with the starting block co-polymer upon an external stimulus to react with the block co-polymer to form the cross-linkable moeities at the chain ends. This external stimulus can be microwave radiation. In some embodiments, the mixture can contain catalyst(s) to change the rate of the reaction.

Cross-linkable moieties can be chosen from but are not limited to acrylates, methacrylates, thiols, carboxyls, esters, hydroxyls, amino groups, isocyanates, azides, isothiocyanates, epoxides, and/or combinations thereof. In a preferred embodiment the cross-linkable moiety is chosen from acrylate(s), methacrylate(s), or combinations thereof. One or more cross-linkable moieties can be used to prepare a mixture of liquid macromers with different reactivity to external stimulation for subsequent polymerization. Multifunctional cross-linkable moieties (moieties that can bond to more than two molecules) can be used to create different networks. In addition, some of these cross-linkable moieties can be used to bond the polymerizable mixture onto the applied surface(s) of medical devices before, during or after polymerization of the liquid, polymerizable mixture. The second monomers are chosen such that the external stimulus and the reaction can lead to the desired cross-linkable moiety at chain ends. For example, methacrylic anhydride can be used to achieve methacrylate moieties on the resultant macromer or acrylic anhydride can be used to achieve acrylate moieties on the resultant macromer.

The duration of the exposure of the mixture to microwave radiation can be anywhere between 1 second to several hours or more. In any of the embodiments, the power of the microwave used can be 50 W, 60 W, 70 W, 80 W, 90 W, 100 W, 200 W, 300 W, 400 W, 500 W, 600 W, 700 W, 800 W, 900 W, 1000 W, 1100 W, 1200 W or more than 1200 W or less than 50 W or in between those wattages. The microwave treatment can be carried under atmospheric pressure, under pressures lower than atmospheric pressure, under partial vacuum, with or without active heating, such radiant heating or convection heating.

In some embodiments, the mixing of the reaction components of the first or second reactions can be done sequentially, or simultaneously. In any of the embodiments, multiple types of the components such as the central moiety or the monomers can be used. Multiple catalysts or solvents can be used.

In any embodiments, the microwave radiation exposure can be performed for a duration of at least 1 second up to several hours, more preferably about 15 seconds to several minutes, most preferable about 30 seconds to 3 minutes. In any of the embodiments, microwave radiation exposure can be performed in a precooled or preheated environment. It can also be performed with active heating or cooling. The heating or cooling can be done at any rate.

In some embodiments, the second monomer is methacrylic anhydride or acrylic anhydride and the corresponding chemical moiety is a methacrylate group (MA) or acrylate group (Acr) and the corresponding macromer is (Bₙ-Aₙ-Bₙ-MA) or (MA-Bₙ-Aₙ-Bₙ-MA) or (Bₙ-Aₙ-Bₙ-Acr) or (Acr-Bₙ-Aₙ-Bₙ-Acr).

In some embodiments, the macromer may be purified by washing with a solvent to remove impurities. Common solvents can be chosen from but are not limited to hexane, diethyl ether, or alcohols such as methanol or ethanol. The washing can be done in multiple steps, each step can use pure solvents or a mixture of solvents. The ratio of block co-polymer to solvent may vary from 1 to 50 volume/volume %, preferably 5-40 volume/volume %, most preferably 10-20 volume/volume %. The temperature of washing may vary from 0 degrees Celsius to 50 degrees Celsius, most preferably 20-30 degrees Celsius. After washing, the solvent may be removed by reduced pressure, elevated temperatures, or a combination of reduced pressure and elevated temperatures. The pressure may range from 20 millibar to 0.5 bar, most preferably 2-5 bar.

After washing, the co-polymer may be stored at temperatures between 0 and 100 degrees Celsius, under atmospheric conditions or under inert gases such as nitrogen, argon, or any other composition of gases. Most preferably, the co-polymer may be stored at temperatures between 20-30 degrees Celsius and under atmospheric conditions.

Storage, Sterilization, Stability

In some embodiments, inhibitor molecule(s) or inhibitor molecule(s) or stabilizers or UV absorbers or antioxidants dissolved or mixed with a solvent may be added to or mixed with the macromer to prevent undesirable polymerization or slow the rate of polymerization or prevent oxidation or otherwise improve the stability of the liquid polymerizable macromer. Non-limiting examples of inhibitors include 4-methoxyphenol, 4-Allyloxy-2-hydroxybenzophenone, 2-(2H-Benzotriazol-2-yl)-4,6-bis(1-methyl-1-phenylethyl)phenol, 2-(2H-Benzotriazol-2-yl)-4,6-di-tert-pentylphenol, 2-(2H-Benzotriazol-2-yl)-6-dodecyl-4-methylphenol, 2-[3-(2H-Benzotriazol-2-yl)-4-hydroxyphenyl]ethyl methacrylate, 2-(2H-Benzotriazol-2-yl)-4-methyl-6-(2-propenyl)phenol, 2-(2H-Benzotriazol-2-yl)-4-(1,1,3,3-tetramethylbutyl)phenol, 2-(4-Benzoyl-3-hydroxyphenoxy)ethyl acrylate, 3,9-Bis(2,4-dicumylphenoxy)-2,4,8,10-tetraoxa-3,9-diphosphaspiro[5.5]undecane, Bis(octadecyl)hydroxylamine powder, 3,9-Bis(octadecyloxy)-2,4,8,10-tetraoxa-3,9-diphosphaspiro[5.5]undecane, Bis(1-octyloxy-2,2,6,6-tetramethyl-4-piperidyl) sebacate, Bis(2,2,6,6-tetramethyl-4-piperidyl) sebacate, 2-tert-Butyl-6-(5-chloro-2H-benzotriazol-2-yl)-4-methylphenol, 2-tert-Butyl-4-ethylphenol, 5-Chloro-2-hydroxybenzophenone, 5-Chloro-2-hydroxy-4-methylbenzophenone, 2,4-Di-tert-butyl-6-(5-chloro-2H-benzotriazol-2-yl)phenol, 2,6-Di-tert-butyl-4-(dimethylaminomethyl)phenol, 3′,5′-Dichloro-2′-hydroxyacetophenone, Didodecyl 3,3′-thiodipropionate, 2,4-Dihydroxybenzophenone, 2,2′-Dihydroxy-4-methoxybenzophenone, 2′,4′-Dihydroxy-3′-propylacetophenone, 2,3-Dimethylhydroquinone, 2-(4,6-Diphenyl-1,3,5-triazin-2-yl)-5-[(hexyl)oxy]-phenol, 5-Ethyl-1-aza-3,7-dioxabicyclo[3.3.0]octane, Ethyl 2-cyano-3,3-diphenylacrylate, 2-Ethylhexyl 2-cyano-3,3-diphenylacrylate, 2-Ethylhexyl trans-4-methoxycinnamate, 2-Ethylhexyl salicylate, 2-Hydroxy-4-(octyloxy)benzophenone, Menthyl anthranilate, 2-Methoxyhydroquinone, Methyl-p-benzoquinone, 2,2′-Methylenebis[6-(2H-benzotriazol-2-yl)-4-(1,1,3,3-tetramethylbutyl)phenol], 2,2′-Methylenebis(6-tert-butyl-4-ethylphenol), 2,2′-Methylenebis(6-tert-butyl-4-methylphenol), Methylhydroquinone, 4-Nitrophenol sodium salt hydrate, Octadecyl 3-(3,5-di-tert-butyl-4-hydroxyphenyl)propionate, Pentaerythritol tetrakis(3,5-di-tert-butyl-4-hydroxyhydrocinnamate), 2-Phenyl-5-benzimidazolesulfonic acid, Poly[[6-[(1,1,3,3-tetramethylbutyl)amino]-s-triazine-2,4-diyl]-[(2,2,6,6-tetramethyl-4-piperidyl)imino]-hexamethylene-[(2,2,6,6-tetramethyl-4-piperidyl)imino], Sodium D-isoascorbate monohydrate, Tetrachloro-1,4-benzoquinone, Triisodecyl phosphite, 1,3,5-Trimethyl-2,4,6-tris(3,5-di-tert-butyl-4-hydroxybenzyl)benzene, Tris(4-tert-butyl-3-hydroxy-2,6-dimethylbenzyl) isocyanurate, Tris(2,4-di-tert-butylphenyl) phosphite, and 1,3,5-Tris(2-hydroxyethyl)isocyanurate, Tris(nonylphenyl) phosphite. The solvent can be aqueous or organic or a mixture. The inhibitor molecule(s) can be dissolved in the liquid, polymerizable mixture or the macromer(s) or the inhibitor can be partially dissolved or the inhibitor may be largely immiscible. The inhibitor can be mixed with the liquid, polymerizable mixture such that the resulting concentration of the inhibitor is from 1 ppm to 10 wt/wt %, or 10 ppm to 1 wt/wt %, more preferably 100 ppm to 1000 ppm.

In some embodiments, the inhibitor molecule is directly blended with the polymeric material by stirring, shaking, physically mixing, or sonication, or using a machine apparatus to mix, shake, stir, or otherwise disperse the initiator molecule.

In any of the embodiments, the macromer or any material or device made from or partially made from the polymerized macromer may be sterilized for use as an implantable medical device. Common methods of sterilization are included but not limited to treatment with ethylene oxide gas, electron-beam radiation, gamma radiation, treatment with peroxides or autoclaving.

Polymerization

In a preferred embodiment of the invention, the macromer is mixed with initiator, which upon exposure to an external stimulus, initiates the polymerization reaction. The initiator molecule(s) may be directly blended with the polymeric material by stirring, shaking, physically mixing, or sonication, or using a machine apparatus to mix, shake, stir, or otherwise disperse the initiator. or using a solvent to dissolve the initiator to disperse it in the macromer. The amount of initiator may be as low as 0.00001 weight percent and up to 10 weight percent, but optimally 0.01-2 weight percent.

In some embodiments, the initiator molecule is a photoinitiator and the polymerization is started by irradiating the macromer and initiator and any other components with light. Non-limiting examples of photoinitiators include phenylbis(2,4,6-trimethylbenzoyl)phosphine oxide, benzoin ethers, benzyl ketals, α-dialkoxyacetophenones, α-hydroxyalkylphenones, α-amino alkylphenonones, acylphophine oxides, peroxides, and acylphosphinates, azobisisobutyronitrile, 1,1′-azobis(cyclohexanecarbonitrile), di-tert-butyl peroxide, benzoyl peroxide, methyl ethyl ketone peroxide, and acetone peroxide. The photoinitiator may be selected based upon the wavelengths of light it is sensitive to, such that the polymerization reaction is initiated at longer or shorter wavelengths of light. In some embodiments, a second initiator molecule or additional initiator molecules can be blended with the photoinitiator.

In some embodiments, the initiator molecule is a thermoinitiator and the polymerization is started by exposing the macromer and initiator mixture to heat. Non-limiting examples of thermoinitiators include ammonium persulfate, tert-Butyl hydroperoxide solution, tert-Butyl peracetate, Cumene hydroperoxide, 2,5-Di(tert-butylperoxy)-2,5-dimethyl-3-hexyne, Dicumyl peroxide, Luperox® 101, Luperox® 224, Luperox® 331M80, Luperox® 531M80, Luperox® A70S, Luperox® A75, Luperox® A98, Luperox® AFR40, Luperox® DDM-9, Luperox® DI, Luperox® LP, Luperox® P, Luperox® TBEC, Luperox® TBH70X, 4,4′-Azobis(4-cyanovaleric acid), 1,1′-Azobis(cyclohexanecarbonitrile), Azobisisobutyronitrile, 2,2′-Azobis(2-methylpropionitrile), 2,2′-Azobis(2-methylpropionitrile). The thermonitiator may be selected based upon its thermoinitiation temperature, such that the polymerization reaction is initiated at higher or lower temperatures. In some embodiments, another molecule or group of molecules may be added with the thermoinitiator to modify the rate of polymerization reaction or increase or decrease its thermoinitiation temperature.

Degradation

As described in Example 16, the solid gel may undergo degradation in the presence of water or bodily fluids or when implanted as part of a surgical procedure. In Example 16, a particular form of the gel loses approximately 45% of its mass over 11 weeks in phosphate-buffered saline.

In some embodiments, modifications to the chemical structure of the macromer may be made to increase or decrease the rate of degradation, for example increasing or decreasing the molecular weight of the hydrophilic block or increasing or decreasing the molecular weight of the degradable block.

In some embodiments, changes to the chemical structure of the macromer may be made to increase or decrease the rate of degradation in response to a different pH.

Therapeutic Loading

In some embodiments, the therapeutic agent is an analgesic. Non-limiting examples of analgesics are bupivacaine, ropivacaine, lidocaine, mepivacaine, prilocaine, levo-bupivacaine, procaine, chloroprocaine, or tetracaine. Salts or free bases of any compounds can be used.

In some embodiments, the therapeutic agent is a non-steroidal anti-inflammatory drug. Non-limiting examples of nonsteroidal anti-inflammatory drugs include aspirin, salicylic acid, ibuprofen, naproxen, ketoprofen, ketorolac, diclofenac, aceclofenac, mefenamic acid, tolfenamic acid, meloxicam, tenoxicam, piroxicam celecoxib, or firocoxib.

In some embodiments, the therapeutic agent is a mixture of two or more therapeutic agents, for example Bupivacaine hydrochloride and Ketorolac tromomethane.

In some embodiments, the therapeutic agent can be mixed with the liquid polymerizable mixture without adding any solvents. In some embodiments, the therapeutic agent may be dissolved or mixed with a solvent such as water or alcohol before blending or mixing the therapeutic agent with the macromer. The solvent may then be subsequently separated from the mixture via application of heat, low pressure, or filtration.

In any embodiments, the blending of the therapeutic agent and/or initiator molecule can be aided by the addition of additives such as surfactants, solvents, or compatabilizers.

In some embodiments, the initiator molecule or source of light radiation or temperature used to initiate the polymerization reaction may be changed to maintain compatibility with a therapeutic agent. For example, ketorolac tromomethane inhibits the polymerization reaction at an irradiation wavelength of 365 nm, but a light source of 395 nm successfully initiates the polymerization reaction as described in Example 20. In some embodiments, the therapeutic agent is not compatible with elevated temperature, so a different initiator may be selected.

Therapeutic Release

In some embodiments, the amount of drug mixed with the macromer may be increased or decreased to increase or decrease the rate of release of the therapeutic agent. Drugs or therapeutic agents or additives can be mixed at a ratio of 0.0001 to 99 wt % of the liquid polymerizable mixture, more preferably at a ratio of 0.1 to 20 wt %, most preferably at a ratio of 1 to 10 wt %.

In some embodiments, the structure of the macromer may be changed in order to increase or decrease the rate of release of the therapeutic agent, or multiple macromers with different structures can be blended to increase or decrease the rate of release of the therapeutic agent.

In some embodiments, multiple therapeutic agents may be mixed into the liquid polymerizable mixture. In some embodiments, the addition of a second therapeutic may increase or decrease the rate of release of the first therapeutic agent, and the first therapeutic agent may increase or decrease the rate of release of the first therapeutic agent.

In some embodiments, the shape or size or surface area of the final material may be modified to increase or decrease the rate of release of the loaded therapeutic agent or therapeutic agents.

Application

In some embodiments. the liquid polymerizable mixture with additives can be applied to another surface such as a biological tissue, a polymeric material, a metal or a ceramic or hybrid materials. The application can be in one step before polymerization or layers can be applied and polymerized on each other. Each of these layers can contain one additive or multiple additives. These additives in the different layers can be the same or different. The thickness of each layer can be several microns to several millimeters, the entire thickness of the polymerized layer on the second surface can be microns to 10 millimeters. The thickness can be uniform or can be modified as desired along the applied surface(s).

In some embodiments, the initiator molecule is incorporated on the application surface placed in contact with the liquid polymerizable mixture. In these embodiments, the polymerization is initiated at the interface between the polymeric material and the application surface. The application surface may be porous, contain holes, or other such geometric features. In some embodiments the liquid polymerizable mixture may infiltrate the holes or pores of the application surface and the polymerization may be initiated such that the holes or pores are filled or partially filled by the resulting polymerized solid gel.

In some embodiments, the macromer can be spread as a thin layer on top of an application surface or device. then the polymerization can be initiated to produce an application surface or device covered with a layer of solid gel.

In some embodiments, the solid gel formed from the polymerized macromer mixture may be physically cut, re-shaped, or machined to produce a solid gel with a different two-dimensional or three-dimensional shape. Other geometric features may be created as the result of cutting or re-shaping or machining, including but not limited to holes, indentations, tapered holes, blunt holes, or screw holes.

In some embodiments, the macromer can be polymerized inside of a container with a specific shape or size. The solid gel will then take the shape of the container.

In some embodiments, the thickness of the layer of solid gel may be modified to ensure a fully-polymerized material is created.

### Definitions

As used herein, the term “monomer” or “macromer” refers to a molecule that may bind covalently to other molecules to form a polymer. The process by which the monomers are combined to form a polymer is called polymerization. Common monomers useful in the methods described herein include, but are not limited to, ethylene oxide, DL-lactide, glycolide, and ε-caprolactone. Macromers are polymeric molecules with reactable monomer end groups.

By “polymerizable liquid” or “polymerizable liquid mixture”, what is meant is at least one chemical substance in the liquid state at room temperature, or mixture of chemical substances in the liquid state at room temperature, which can become a solid or gel upon polymerization. In the liquid state, the substance or mixture can flow and/or change shape upon mechanical disturbance. The liquid mixture can be mainly a liquid; that is, there can be gaseous or solid components dispersed in the liquid; as long as the mixture is able to flow and is homogeneous, it is termed a polymerizable liquid mixture. Similarly, all components can be dissolved in each other, thus the liquid could be a solution or a multi-component solution. The components of the mixture can have primary functions such as being a cross-linkable macromer, an initiator, or a cross-linking enhancer but can also aid in the solvation of other components. The liquid polymerizable mixture can also have non-polymerizable or non-reactable polymeric components, for example oligomers of polyethylene glycol. The liquid polymerizable mixture may also be referred to as a ‘liquid polymerizable macromer mixture’ and can have non-reactive polymers or additives.

As used herein, the term “polymer segment” means and includes a grouping of multiple monomer units of the same type (i.e. a homopolymer segment) or of different types (i.e. a co-polymer segment) of constitutional units joined together into a continuous polymer chain.

As used herein, the term “polymer block” means and includes a grouping of multiple monomer units of the same type (i.e. a homopolymer block) or of different types (i.e. a co-polymer block) of constitutional units joined together into a continuous polymer chain that forms part of a larger polymer of even greater length.

As used herein, the term “block co-polymer” means and includes a polymer composed of chains where each chain is composed of two or more polymer blocks as defined above. A block co-polymer may be represented herein by (Aₙ-Bₘ), where A and B represent monomers and n and m each represent the number of repeats.

As used herein, the term “random co-polymer” means and includes a polymer chain formed from two different monomers arranged in a pattern having no particular order to form a polymer segment. Random co-polymers may be represented by (Aₙ-r-Cₚ), where the capital letters A and C represent monomers, n and p each represent the number of repeats, and r represents that the sequence of A and C monomers is random and has no particular order.

As used herein, the term “chemical moiety” represents a grouping of atoms in a specific arrangement which form covalent chemical bonds in a specific sequence and type.

By “connecting moiety” what is meant is a molecule or part of molecule that connects biodegradable moiety with biodegradable moiety, biodegradable moiety with cross-linkable moiety, and/or cross-linkable moiety with cross-linkable moiety. Such connecting moiety(ies) can be chosen from the group of but are not limited to polyethylene glycol, polyethylene oxide, polypropylene glycol, 1,6-hexanediol, 2,2,6,6-Tetrakis(hydroxymethyl)cyclohexanol, ethylene glycol, cyanuric acid. Such connecting moieties consist of a mixture of one or more types and consists of a mixture of different molecular weight distributions. In a preferred embodiment, the connecting moiety is liquid at room temperature. In one embodiment, the connecting moiety can be a mixture of polyethylene glycol and propylene glycol. In another embodiment, the connecting moiety can be a mixture of polyethylene glycol with average molecular weight of 200 and polyethylene glycol with average molecular weight of 400. In another embodiment, the connecting moiety has a random distribution(s) of weight average molecular weight polyethylene glycol. In the preferred embodiment, the connecting moiety can be polyethylene glycol with weight average molecular weight of 200 (PEG 200). In another preferred embodiment, the connecting moiety can be polyethylene glycol with weight average molecular weight of 400 (PEG 400).

By “biodegradable moiety”, what is meant is a molecule or part of molecule that can be degraded (e.g. cleaved and/or destroyed and/or decomposed inside the body) and eliminated by the body. The cleaving, destroying, or decomposing can be through hydrolysis, enzymatic degradation, modification by the liver, excretion by the kidney(s) and/or combinations thereof. Modification by the liver means the changing of the degraded polymer by the liver. Such biodegradable moiety can be but not limited to poly(lactide) (PLA), poly(glycolide) (PGA), poly(epsilon-caprolactone) (PCA), poly(dioxane) (PDA), poly(trimethylene carbonate) (PTMC), and combinations thereof. In one embodiment, the biodegradable moiety is polyglycolide. In another embodiment, the biodegradable moiety is polylactide-co-polyglycolide. In another embodiment, the biodegradable moiety is polytrimethylene carbonate-co-poly(epsilon-caprolactone). In a preferred embodiment, the biodegradable moiety is polylactide with length of 1-8 lactoyl groups. In another preferred embodiment, the biodegradable moiety is polyglycolide with length of 1-8 glycolyl groups. In another preferred embodiment, the biodegradable moiety is polycaprolactone with length of 1-8 epsilon-caprolactone groups. In certain preferred embodiments, the biodegradable moiety is a polylactide with 2-4 lactoyl groups.

By “cross-linkable moiety”, what is meant is a molecule or part of a molecule that can form one or more new bond(s) (covalent and/or non-covalent) with another molecule, preferably a macromonomer to create a network of molecule(s) and/or macromonomers. Such cross-linkable moieties can comprise acrylate(s), methacrylate(s), thiols, carboxyls, hydroxyls, amino groups, isocyanates, azides, isothiocyanates, epoxides, and/or combinations thereof). In a preferred embodiment, the cross-linkable moiety comprises acrylate(s), methacrylate(s), or combinations thereof. In more preferred embodiment, the cross-linkable moiety comprises an acrylate group.

As used herein, the term “catalyst” or “catalyst molecule” represents a chemical compound that drastically accelerates the rate of a chemical reaction without changing the reaction products. For example, stannous octoate is a typical catalyst used for the ring opening polymerization of lactide with polyethylene glycol. Catalysts for the central moiety and degradable moiety reactions initiated by microwave radiation and catalysts for the degradable moiety and cross-linkable moiety reactions can be chosen from the following list but are not limited to it: stannous octoate, 1-(1-Adamantyl)-3-(2,4,6-trimethylphenyl)imidazolinium chloride, Bis(cyclopentadienyl)dimethylzirconium(IV), ichloro[1,3-bis(2,6-isopropylphenyl)-2-imidazolidinylidene](benzylidene)(tricyclohexylphosphine)ruthenium(II), Dichloro[1,3-Bis(2-methylphenyl)-2-imidazolidinylidene](benzylidene)(tricyclohexylphosphine)ruthenium(II), Dichloro[1,3-bis(2,4,6-trimethylphenyl)-2-imidazolidinylidene][3-(2-pyridinyl)propylidene]ruthenium(II), Diethylmethoxyborane, Dysprosium(III) trifluoromethanesulfonate, Grubbs Catalyst® C571, Grubbs Catalyst® C711, Grubbs Catalyst® C7827, Grubbs Catalyst® C833, Grubbs Catalyst® C859, Grubbs Catalyst® 1^{st} generation, Grubbs Catalyst® 2^{nd} generation, Grubbs Catalyst® 3^{rd} generation, Hoveyda-Grubbs Catalyst® 1^{st} generation, Hoveyda-Grubbs Catalyst® 2^{nd} generation, Methyltriphenylphosphonium chloride, Neodymium(III) trifluoromethanesulfonate, or Praseodymium(III) trifluoromethanesulfonate.

As used herein, the term “photoinitiator” represents a chemical compound that can produce radical species and/or promote radical reactions when exposed to light irradiation. Common photoinitiators useful in the methods, compositions, and systems described herein include, but are not limited to, benzoin ethers, benzyl ketals, α-dialkoxyacetophenones, α-hydroxyalkylphenones, α-amino alkylphenonones, acylphophine oxides, peroxides, and acylphosphinates. azobisisobutyronitrile, 1,1′-azobis(cyclohexanecarbonitrile), di-tert-butyl peroxide, benzoyl peroxide, methyl ethyl ketone peroxide, and acetone peroxide. An exemplary photoinitiator is phenylbis(2,4,6-trimethylbenzoyl)phosphine oxide.

As used herein, the term “thermoinitiator” represents a chemical compound that can produce radical species and/or promote radical reactions when exposed to heat or elevated to a certain temperature. Common classes of thermoinitiators include azo compounds, inorganic peroxides, and organic peroxides. Some non-limiting examples of thermoinitiators include 4,4′-Azobis(4-cyanovaleric acid), 1,1′-Azobis(cyclohexanecarbonitrile), Azobisisobutyronitrile, Ammonium persulfate, Hydroxymethanesulfinic acid, Potassium persulfate, Sodium persulfate, tert-Butyl hydroperoxide, tert-Butyl peracetate, Cumene hydroperoxide, 2,5-Di(tert-butylperoxy)-2,5-dimethyl-3-hexyne, Dicumyl peroxide, 2,5-Bis(tert-butylperoxy)-2,5-dimethylhexane, 2,4-Pentanedione peroxide, 1,1-Bis(tert-butylperoxy)-3,3,5-trimethylcyclohexane, Benzoyl peroxide, 2-Butanone peroxide, tert-Butyl peroxide, tert-Butyl peroxybenzoate, tert-Butylperoxy 2-ethylhexyl carbonate, tert-Butyl hydroperoxide, and hydrogen peroxide.

As used herein, the term “microwave radiation” represents electromagnetic radiation with wavelengths ranging from one meter to one millimeter. Microwave irradiation can be done at temperatures between 0 and 100 degrees Celsius, but preferably between 20 and 30 degrees Celsius. It can be done in inert atmosphere or in environments with varying concentrations of gases such as nitrogen or oxygen or mixtures thereof. By ‘microwave oven’ is meant an appliance that contains a cavity into which microwaves are sent, causing items in the cavity to be irradiated with microwave radiation. The power of the microwave used can be 50 W, 60 W, 70 W, 80 W, 90 W, 100 W, 200 W, 300 W, 400 W, 500 W, 600 W, 700 W, 800 W, 900 W, 1000 W, 1100 W, 1200 W or more than 1200 W or less than 50 W or in between those wattages. The microwave treatment can be carried under atmospheric pressure, under pressures lower than atmospheric pressure, under partial vacuum, with or without active heating, such radiant heating or convection heating. The intensity of microwave radiation may be homogeneous throughout the cavity, or certain locations within the cavity may experience higher intensity of radiation than others. The cavity may be designed to intentionally provide uniform or non-uniform intensity of microwave radiation by changing the shape of the cavity or the source of microwave radiation or by adding elements to the cavity that reflect microwave radiation in a specific direction or directions.

As used herein, the term “macromer” represents a polymer chain containing a chemical moiety at one or both ends of the polymer chain which can undergo further chemical reactions to either extend the polymer chain or form a network of polymer chains.

By “applying to the surface or surfaces of a medical device”, what is meant is contacting one or more parts or all of the surfaces of a medical device with the liquid, polymerizable mixture by for example, filling pre-formed reservoirs on the surface(s) of the medical device with mixture, painting surface(s) of medical device with mixture, spraying surface of medical device by mixture and combinations thereof. The adhesion between the liquid, polymerizable mixture and the medical device surface can be enhanced by using an adhesive. Alternatively, the bond between the medical device surface and the drug-eluting polymer resulting from the polymerization of the liquid, polymerizable mixture on the surface of medical device can be enhanced by mechanical interfacing using design features on the medical device such as pores or locking features. This can also be enhanced by compressing or loading of the drug-eluting polymer onto the medical device surface. In one preferred embodiment, the polymerizable liquid is added as a layer on one or more reservoir(s) of a medical device and then polymerized. More preferably, the polymerizable liquid is added on the surface of a medical device and then polymerized. The liquid polymerizable mixture can be applied to the surface or surface(s) of implantable components such as tissue allograft(s). This application as well as further polymerization can be done before or after the allograft has been implanted. When the liquid polymerizable mixture is not applied to the surface or surfaces of a medical device, it can be applied directly at the site of treatment. Examples for the site of treatment can be the peri-prosthetic tissue around a fracture or a joint implant or degenerative disc(s) or a skin wound.

In one embodiment, the surface(s) include the backside or side walls of the tibial insert or tibial base plate used in total knee replacement. In another embodiment, the surface is along the femoral stem, on the rim of acetabular cup, backside of the acetabular cup as components of total hip replacement. In another embodiment, the surface is on the pacemaker pulse generator, inside or outside lumen of catheter.

In one embodiment, surface of the medical device can have reservoir(s). By “reservoir”, what is meant is any surface feature that allows at least temporary containment of the liquid, polymerizable liquid. In a preferred embodiment, the medical device has reservoir(s) that are previously machined and the liquid, polymerizable mixture is added into these reservoirs. In another embodiment, reservoir(s) can be formed on the medical device at the time of implantation; for instance in the operating room. In another embodiment, reservoir(s) are created in situ at the time of implantation by medical professionals such as but not limited to physician, nurses, physician assistant. In another preferred embodiment, the reservoirs are pre-formed or formed at the time of implantation on the backside of tibial inserts, sidewalls of tibial insert, rims of acetabular cups, backside of acetabular cups, femoral stems, tibial baseplates, knee femoral components, pacemakers, implantable cardioverter defibrillators, or catheters. In another embodiment, the reservoir is the space above the screw head inside a screw-hole of an acetabular shell or a tibial baseplate.

By “medical device”, what is meant is an instrument, apparatus, implement, machine, implant or other similar and related article intended for use in the diagnosis, treatment, mitigation, cure, or prevention of disease in humans or other animals. An “implantable device” is a medical device intended to be implanted in contact with the human or other animal for a period of time. The primary function of the implantable medical device can be monitoring signals, delivering drugs or the replacement of tissues or the function of tissues among other functions. The implantable medical device can be permanent or can be removed after a period of time. A medical device can be made out of metal, polymer, ceramic or a combination thereof. A medical device can also contain organic tissue or modified organic tissue. Examples of implantable medical devices are acetabular shells, acetabular cups, femoral heads, modular or nonmodular femoral necks, tibial inserts, tibial baseplates, fixation pins, fracture plates, rods, screws, shoulder implants, pacemakers, ventricular assist devices, implantable cardioverter defibrillators. In an embodiment, the medical device is a urinary catheter, a central venous catheter, a femoral central venous catheter. In one embodiment, the medical device can be made of titanium alloy such as TiAl6V4, cobalt chrome alloy, poly ether ether ketone, ultrahigh molecular polyethylene, polyurethane, and combinations thereof. Fixation devices can be used collectively to indicate different components used in the fixation of a fracture, for example fracture plates, fixation pins and screws.

As used herein, the term “gel” or “gel network” represents a non-fluid network of polymer chains formed from a previously fluid solution of polymer chains and possibly including other additives. A gel may be formed by a network of polymer chains joined through covalent bonds, nonlinear polymerization, or through non-covalent aggregation of polymer chains or segments. The process by which a fluid solution of polymer chains and possibly including other additives are combined to form a gel network is called gelation. By way of non-limiting example, gelation may be caused by a chemical reaction initiated by light, heat, change of temperature, or other radiation. Optionally, a gel may have its volume expanded by a fluid or solvent, e.g. water.

In the representations used herein, (e.g. (Aₙ), B, (Aₙ-Bₘ-Aₙ), (R-Aₙ-Bₘ-Aₙ-R), ((Aₙ-r-Cₚ)-Bₘ-Aₙ-(Aₙ-r-Cₚ)), and (R-(Aₙ-r-Cₚ)—Bₘ-Aₙ-(Aₙ-r-Cₚ)—R)), the capital letters A, B, and C represent monomers. Subscript lowercase letters immediately adjacent to a capital letter indicates the number of repeats of the monomer of that type in a sequence. The capital letter R represents a chemical moiety. Segments or blocks of monomers are set apart by parentheses. Lowercase letters between capital letters and set apart by hyphens indicate an ordering arrangement between the monomers in the sequence. Specifically, “r” indicates that the ordering is random. Hyphens setting apart polymer segments or polymer blocks or chemical moieties indicate that the adjoining polymer segments or blocks or chemical moieties are connected in sequence.

As used herein, the term “degradable” or “degradable material” means that the material decomposes through either physical means or chemical means or both physical and chemical means at a certain period of time after the material is implanted as a medical device. By “biodegradation” it is meant to include cleaving, destroying, or decomposing through hydrolysis, enzymatic degradation, biological modification by the liver, excretion by the kidney(s) and combinations of these modes of degradation. Biological modification by the liver means the changing of the chemical structure of the degraded polymer by the liver. As a result, the drug eluting polymer disappears in a certain period after implantation and therefore is no longer a potential surface for colonization by bacteria. The time that it takes for the material to degrade may be as short as one minute or as long as ten years or any length of time between one minute and ten years. The material degradation may be measured by a loss of mass of material, loss of volume of material, decrease in the mechanical stiffness of the material, or change in the molecular structure of the material.

As used herein, the term “additive” is any chemical compound or mixture of chemical compounds that is intended to improve upon the ease of processing or performance of the final material that is mixed or blended in with the macromer before it is polymerized into a solid gel. Additives may include but are not limited to surfactants, solvents, other monomers, macromers, polymers, acids, bases, salts, ceramics, particles or particulate materials, fibers, organic molecules, or inorganic compounds.

As used herein, the term “therapeutic agent” represents any molecule or mixture of molecules known in the art to have a biological effect on a human or animal upon ingestion, injection, or implantation such as a drug (e.g. a molecule approved by the U.S. Food and Drug Administration as provided in the Code of Federal Regulations (C.F.R.)). Common classes of therapeutic agents are antibiotics, anti-inflammatory agents, anesthetic agents, anticoagulants, hormone analogs, contraceptives, vasodilators, vasoconstrictors, or other molecules classified as drugs in the art.

As used herein, the term “inhibitor” or “polymerization inhibitor” represents any molecule or mixture of molecules known in the art to prevent or slow the rate of a polymerization reaction. Some non-limiting examples of polymerization inhibitors are 4-tert-butylpyrocatechol, tert-Butylhydroquinone, 1,4-Benzoquinone, 6-tert-Butyl-2,4-xylenol, 2,6-Di-tert-butyl-p-cresol, 2,6-Di-tert-butylphenol, 1,1-Diphenyl-2-picrylhydrazyl, Hydroquinone, 4-Methoxyphenol, and phenothiazine.

### EXAMPLES

### Example 1

### Synthesis of MA-PLA₄-PEG_{4.5}-PLA₄-MA

1.53 g of PEG with a molecular weight of 200 g/mol was mixed with 4.45 g of DL lactide and 33 mg of stannous octoate. The mixture was heated with microwave radiation for 1.5 minutes to produce PLA₄-PEG_{4.5}-PLA₄. 6.94 g of methacrylic anhydride was added and the mixture was heated with microwave radiation for 1.5 minutes to produce MA-PLA₄-PEG_{4.5}-PLA₄-MA

### Example 2

### Synthesis of MA-PLA₄-PEG₉-PLA₄-MA

2.51 g of PEG with a molecular weight of 400 g/mol was mixed with 3.6 g of DL lactide and 55 mg of stannous octoate. The mixture was heated with microwave radiation for 2 minutes to produce PLA₄-PEG₉-PLA₄. 2.59 g of methacrylic anhydride was added and the mixture was heated with microwave radiation for 2 minutes to produce MA-PLA₄-PEG₉-PLA₄-MA.

### Example 3

### Synthesis of MA-PLA₄-PEG₁₄-PLA₄-MA

2.5 g of PEG with a molecular weight of 600 g/mol was mixed with 2.41 g of DL lactide and 62 mg of stannous octoate. The mixture was heated with microwave radiation for 2 minutes to produce PLA₄-PEG₁₄-PLA₄. 2.31 g of methacrylic anhydride was added to PLA₄-PEG₁₄-PLA₄. and the mixture was heated with microwave radiation for 2 minutes to produce MA-PLA₄-PEG₁₄-PLA₄-MA.

### Example 4

### Synthesis of MA-PLA₂-PEG₃₄-PLA₂-MA

11.29 g of PEG with a molecular weight of 1500 g/mol was mixed with 2.26 g of DL lactide and 60 mg of stannous octoate. The mixture was heated with microwave radiation for 2 minutes to produce PLA₂-PEG₃₄-PLA₂.

7.71 g of PLA₂-PEG₃₄-PLA₂ was added to 3.42 g of methacrylic anhydride and the mixture was heated with microwave radiation for 2 minutes to produce MA-PLA₂-PEG₁₄-PLA₂-MA.

### Example 5

### Synthesis PCL₄-PEG₂₃-PCL₄

5.09 g of PEG with a molecular weight of 1000 g/mol was mixed with 116.2 mg of stannous octoate and 4.65 g ε-caprolactone. The mixture was heated with microwave radiation for 2 minutes to produce PCL₄-PEG₂₃-PCL₄.

### Example 6

### Incorporation of Bupivacaine.HCl into MA-PLA₄-PEG₉-PLA₄-MA and Gelation

MA-PLA₄-PEG₉-PLA₄-MA was mixed with Bupivacaine Hydrochloride to produce Bupivacaine-loaded MA-PLA₄-PEG₉-PLA₄-MA. Phenylbis(2,4,6-trimethylbenzoyl)phosphine oxide was added to Bupivacaine-loaded MA-PLA₄-PEG₉-PLA₄-MA and irradiated with ultraviolet light with a wavelength of 365 nm for 5 minutes to produce a solid gel form of bupivacaine-loaded MA-PLA₄-PEG₉-PLA₄-MA.

### Example 7

### Gelation of MA-PLA₂-PEG₃₄-PLA₂-MA with 2-Hydroxy-4′-(2-hydroxyethoxy)-2-methylpropiophenone

1.32 g MA-PLA₂-PEG₃₄-PLA₂-MA was mixed with 17.4 mg of 2-Hydroxy-4′-(2-hydroxyethoxy)-2-methylpropiophenone and irradiated with ultraviolet light for 1 minute to produce a solid gel form of MA-PLA₂-PEG₃₄-PLA₂-MA.

### Example 8

### Incorporation of Bupivacaine.HCl into MA-PLA₄-PEG₁₄-PLA₄-MA and Gelation

MA-PLA₄-PEG₁₄-PLA₄-MA was mixed with Bupivacaine Hydrochloride to produce Bupivacaine-loaded MA-PLA₄-PEG₁₄-PLA₄-MA. Phenylbis(2,4,6-trimethylbenzoyl)phosphine oxide was added to Bupivacaine-loaded MA-PLA₄-PEG₁₄-PLA₄-MA and irradiated with ultraviolet light with a wavelength of 365 nm for 5 minutes to produce a solid gel form of bupivacaine-loaded MA-PLA₄-PEG₁₄-PLA₄-MA.

### Example 9

### Bupivacaine Hydrochloride Release from Bupivacaine-Loaded MA-PLA₄-PEG₉-PLA₄-MA Gel and Bupivacaine-Loaded MA-PLA₄-PEG₁₄-PLA₄-MA Gel

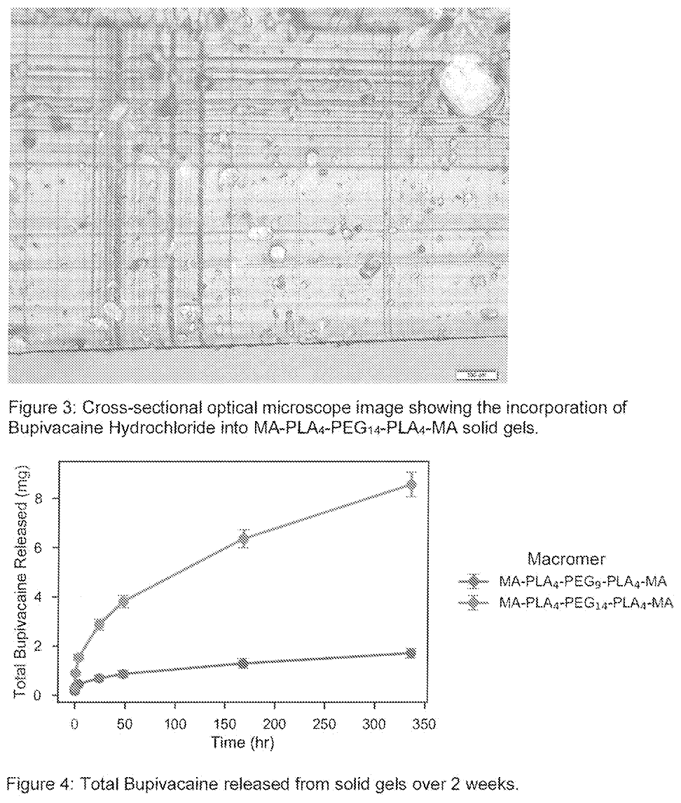

Bupivacaine-loaded MA-PLA₄-PEG₉-PLA₄-MA solid gel networks and Bupivacaine-loaded MA-PLA₄-PEG₁₄-PLA₄-MA gel networks with dimensions 3 mm×5 mm×20 mm and total Bupivacaine Hydrochloride content 5% by weight were loaded into 2 mL phosphate-buffered saline. At pre-selected time points, the phosphate-buffered saline was replaced and the concentration of Bupivacaine in the solution was measured by high-performance liquid chromatography. The results of this experiment are in FIG. 3.

### Example 10

### Polymerization of MA-PLA₄-PEG₁₄-PLA₄-MA on Top of an Initiator-Containing Substrate

0.52 g of 4-hydroxybenzophenone was mechanically mixed with 103.55 g ultra-high molecular weight polyethylene powder (GUR 1020 resin, Celanese Corp., Irving, Tex.) to create a blend with 0.5% 4-hydroxybenzophenone. 13.2 g of this blend was consolidated into a solid material via compression molding at 20 MPa pressure and 180° C. A layer of MA-PLA₄-PEG₁₄-PLA₄-MA was added to the surface and irradiated for 5 minutes with UV light, forming a solid gel layer of the MA-PLA₄-PEG₁₄-PLA₄-MA.

### Example 11

### Fourier-Transform Infrared Spectroscopy Analysis of Macromer and Solid Gel

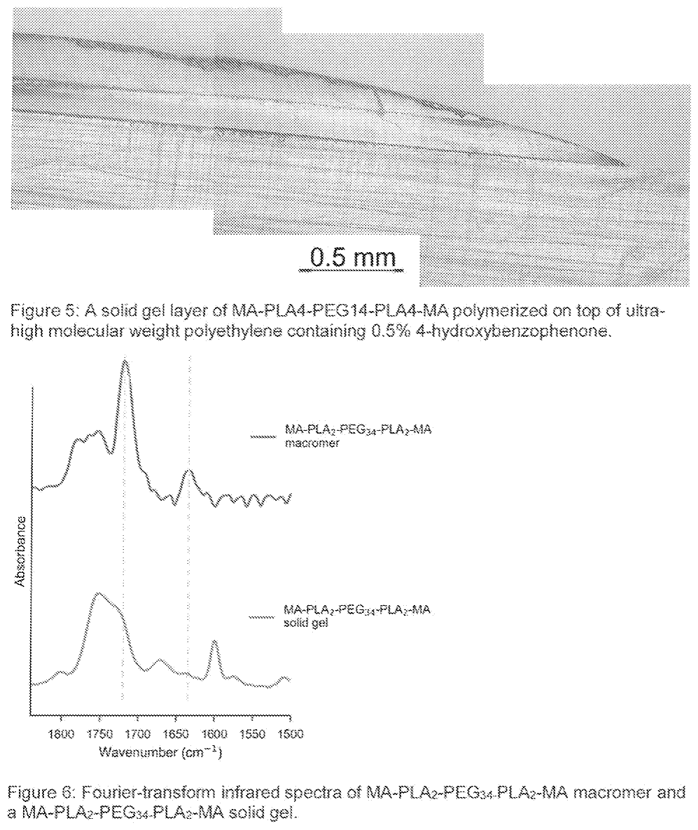

MA-PLA₂-PEG₃₄-PLA₂-MA was prepared as described in Example 3, and solid gel MA-PLA₂-PEG₃₄-PLA₂-MA was prepared in a similar manner to Example 6, using 447 mg of MA-PLA₂-PEG₃₄-PLA₂-MA, 44.7 μL water, and 33.1 mg of 2-Hydroxy-4′-(2-hydroxyethoxy)-2-methylpropiophenone. The sample was irradiated with broad-spectrum UV light for 10 seconds. Both samples were examined using Fourier Transform Infrared Spectroscopy using attenuated total reflection mode. The results as shown in FIG. 5 show a decrease in the absorbance of unsaturated ester chemical moieties (wavenumber 1720 cm⁻¹) and a decrease in the absorbance of carbon-carbon double bonds (wavenumber 1630 cm⁻¹).

### Example 12

### Nuclear Magnetic Resonance Spectroscopy of PCL₄-PEG₂₃-PCL₄

Approximately 100 mg of PCL₄-PEG₂₃-PCL₄ was dissolved in deuterated chloroform and the proton nuclear magnetic resonance spectrum was taken using a Bruker AVANCE III 400 MHz spectrometer (Massachusetts Institute of Technology, Cambridge, Mass.). The results of this experiment are shown in FIG. 6.

### Example 13

### Nuclear Magnetic Resonance Spectroscopy of PLA₄-PEG₂₃-PLA₄

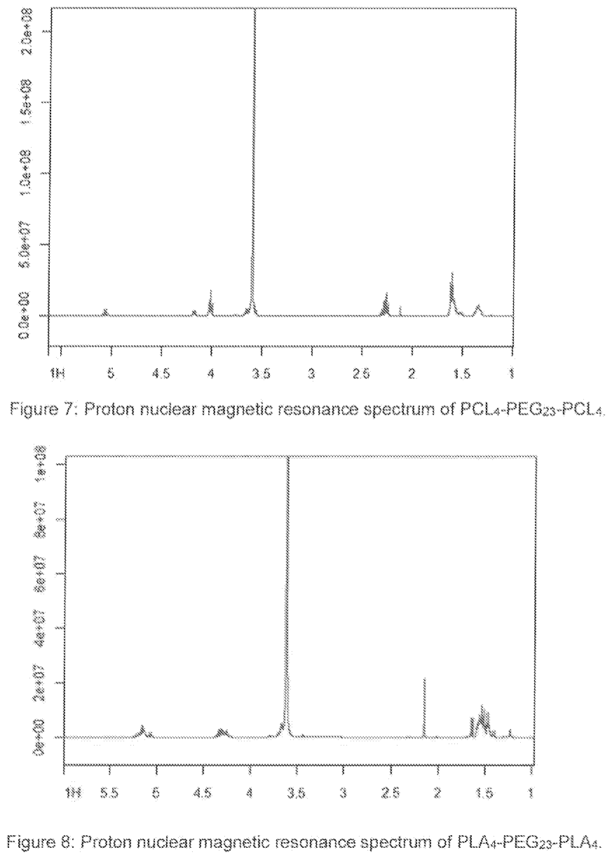

Approximately 100 mg of PLA₄-PEG₂₃-PLA₄ was dissolved in deuterated chloroform and the proton nuclear magnetic resonance spectrum was taken using a Bruker AVANCE III 400 MHz spectrometer (Massachusetts Institute of Technology, Cambridge, Mass.). The results of this experiment are shown in FIG. 7.

### Example 14

### Nuclear Magnetic Resonance Spectroscopy of MA-PLA₄-PEG₂₃-PLA₄-MA

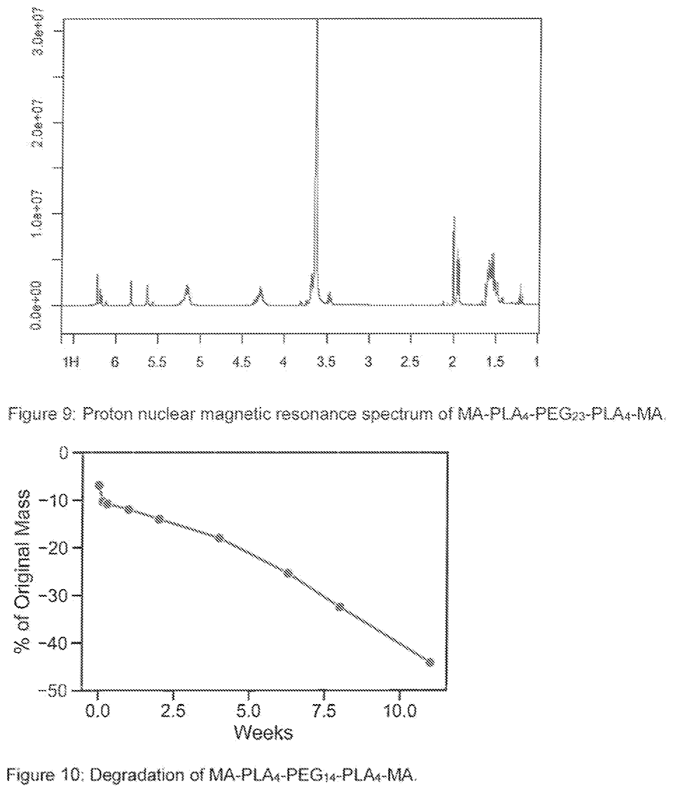

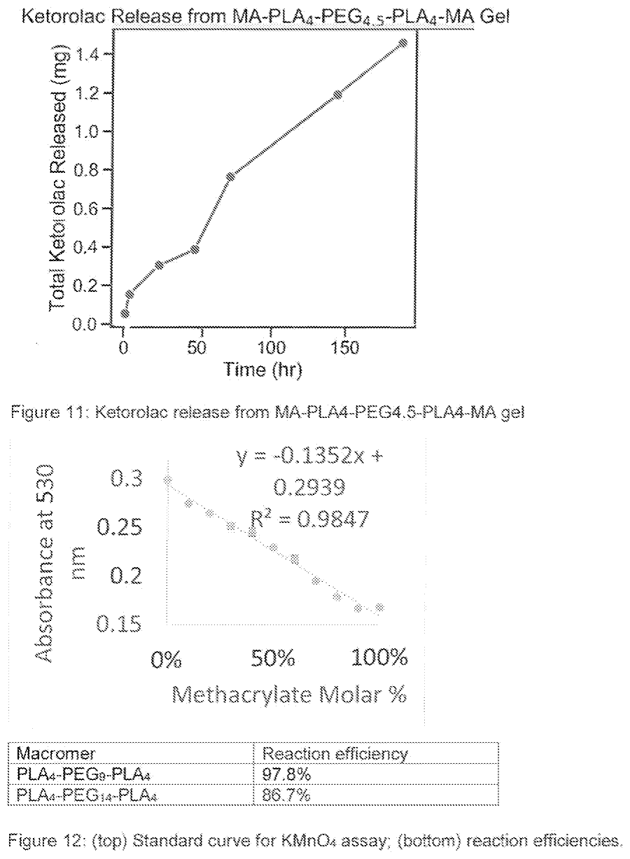

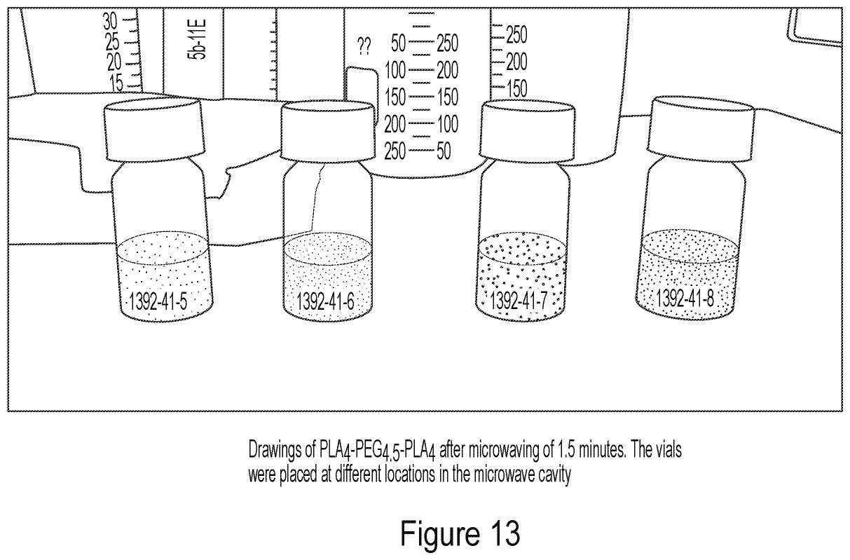

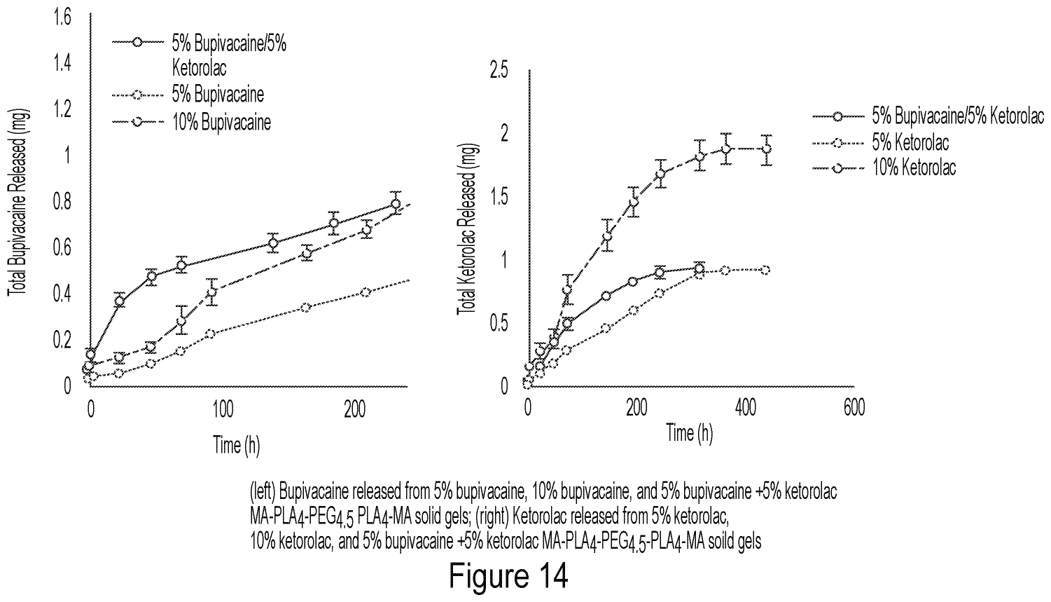

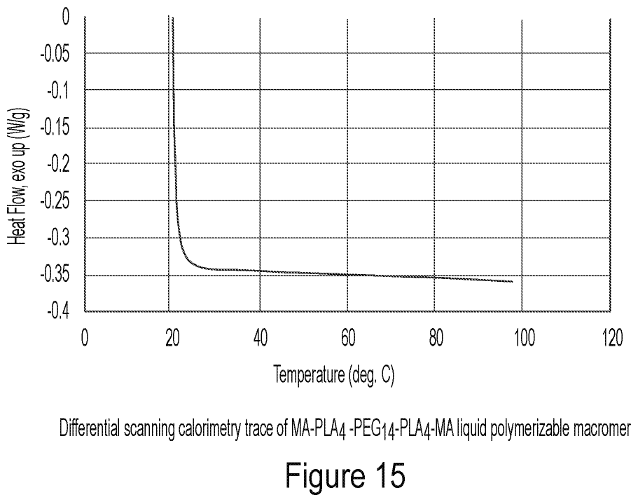

Approximately 100 mg of MA-PLA₄-PEG₂₃-PLA₄-MA was dissolved in deuterated chloroform and the proton nuclear magnetic resonance spectrum was taken using a Bruker AVANCE III 400 MHz spectrometer (Massachusetts Institute of Technology, Cambridge, Mass.). The results of this experiment are shown in FIG. 8.

### Example 15

### Rheological Measurement of MA-PLA₄-PEG₁₄-PLA₄-MA Solid Gel

3.39 g MA-PLA₄-PEG₁₄-PLA₄-MA was mixed with 7.4 mg phenylbis(2,4,6-trimethylbenzoyl)phosphine oxide and 0.84 g of 1% polyethylene glycol sorbitan monolaurate in water. Approximately 0.5 g of this mixture was loaded on to the bottom plate of a TA Instruments AR2000ex rheometer, modified to include a 395 nm UV light source. The upper plate of the rheometer was brought down to a gap of 1 mm, and the macromer mixture was irradiated with the 395 nm light for 3.5 minutes, forming a solid gel. After this, the rheometer was used in oscillatory mode to measure the stiffness of the resulting solid gel. The storage modulus G′, a measure of the stiffness of a material known in the art, was measured to be 180 kPa at an angular frequency of 10 radians per second.

### Example 16

### Degradation of MA-PLA₄-PEG₁₄-PLA₄-MA Solid Gel

MA-PLA₄-PEG₉-PLA₄-MA was mixed with phenylbis(2,4,6-trimethylbenzoyl)phosphine oxide and exposed to ultraviolet light to create a solid gel. Cylindrical samples with approximately 5 mm diameter and 1 mm thickness were created and placed in phosphate-buffered saline and maintained at a temperature of 37° C. The material degradation was measured gravimetrically after dehydrating the samples. The results are shown in FIG. 9.

### Example 17

### Ketorolac Tromethamine Release from Bupivacaine-Loaded MA-PLA₄-PEG_{4.5}-PLA₄-MA Gel

Ketorolac tromethamine-loaded MA-PLA₄-PEG_{4.5}-PLA₄-MA solid gel networks containing 10 weight percent ketorolac tromethamine were cut into cylinders with 1 mm thickness and approximately 5 mm diameter. Samples were loaded into 1 mL phosphate-buffered saline. At pre-selected time points, the phosphate-buffered saline was replaced and the concentration of ketorolac in the solution was measured by spectrophotometery. The results of this experiment are in FIG. 10.

### Example 18

### Efficiency of the Chemical Reaction Between the Second Monomer and the Block Copolymer

PLA₄-PEG₉-PLA₄ liquid polymerizable macromer was prepared by reacting 5 g PEG with a molecular weight of 400 g/mol with 7.2 g D,L lactide and 0.1 g stannous octoate by microwaving for 1.5 minutes. 4.24 g of methacrylic anhydride was added, and the mixture was microwaved for 1.5 minutes more to create MA-PLA₄-PEG₉-PLA₄-MA. The mixture was washed with hexane and diethyl ether.

PLA₄-PEG₁₄-PLA₄ was prepared by reacting 5 g PEG with a molecular weight of 600 g/mol with 4.8 g D,L lactide and 0.1 g stannous octoate by microwaving for 1.5 minutes. 4.24 g of methacrylic anhydride was added, and the mixture was microwaved for 1.5 minutes more to create MA-PLA₄-PEG₁₄-PLA₄-MA. The mixture was washed with hexane and diethyl ether.

The efficiency of the chemical reaction between the second monomer (methacrylic anhydride) and the block colpolymer was quantified using a potassium permanganate (KMnO₄) assay. Synthesized macromers were dissolved to 10.9 mM in a 1:1 acetone/de-ionized water solution, then diluted with de-ionized water to 0.14 mM. 100 μL of this solution was mixed with 100 μL of 0.475 mM KMnO₄. The absorbance at 530 nm was read with a BioTek Synergy H1 Plate Reader. A standard curve was generated using mixtures of hydroxyl-terminated PEG (400 g/mol) and PEG dimethacrylate (575 g/mol). FIG. 12 shows the reaction efficiencies for MA-PLA₄-PEG₉-PLA₄-MA and MA-PLA₄-PEG₁₄-PLA₄-MA.

### Example 19

### Inhomogeneity of the Microwave Cavity

In four separate vials, 2.5 g of PEG with a molecular weight of 200 g/mol was mixed with 7.21 g of D,L lactide and 0.05 g stannous octoate. The vials were placed at different locations in the microwave cavity and microwaved for 1.5 minutes. The vials displayed different colors, indicating a higher or lower extent of the polymerization of lactide (FIG. 13).

### Example 20

### Drug Release from Mixed-Drug Solid Gels

2.5 g of PEG with a molecular weight of 200 g/mol was mixed with 7.21 g of D,L lactide and 0.05 g stannous octoate. The vials were placed at different locations in the microwave cavity and microwaved for 1.5 minutes. 4.24 g of methacrylic anhydride was added and the mixture was microwaved for 1.5 minutes more. The mixture was washed with hexane and diethyl ether to produce MA-PLA₄-PEG_{4.5}-PLA₄-MA liquid polymerizable macromer. The liquid polymerizable macromer was mixed with phenylbis(2,4,6-trimethylbenzoyl)phosphine oxide to a total content of 0.05 wt %. Then, the liquid polymerizable macromer was mixed with bupivacaine hydrochloride and/or ketorolac tromethamine powders to create 5 wt % bupivacaine hydrochloride, 5 wt % ketorolac tromethamine, and 5 wt % bupivacaine hydrochloride +5 wt % ketorolac tromethamine. The mixtures were irradiated with UV light to create solid gels. The drug release from these gels was measured (FIG. 14).

### Example 21

### Stability at Elevated Temperature

2.50 g of PEG with a molecular weight of 600 g/mol was mixed with 2.41 g D,L lactide and 0.06 g of stannous octoate. The mixture was microwaved for 1 minute. 1.36 mL of methacrylic anhydride was added, and the mixture was microwaved for 1 more minute. The mixture was then washed with hexane and diethyl ether, producing MA-PLA₄-PEG₁₄-PLA₄-MA liquid polymerizable macromer. The liquid polymerizable macromer was mixed with 4-methoxyphenol was at a final concentration of 100 ppm and phenylbis(2,4,6-trimethylbenzoyl)phosphine oxide to a final concentration of 0.28 ppm. The mixture was heated in a differential scanning calorimeter from 20 degrees Celsius to 100 degrees Celsius at a rate of 10 degrees Celsius per minute. No peaks were observed in the heat flow curve (FIG. 15), demonstrating that the mixture is stable at elevated temperature.

### Example 22

### Stability at Elevated Temperature

2.50 g of PEG with a molecular weight of 600 g/mol was mixed with 2.41 g D,L lactide and 0.06 g of stannous octoate. The mixture was microwaved for 1 minute. 1.36 mL of methacrylic anhydride was added, and the mixture was microwaved for 1 more minute. The mixture was then washed with hexane and diethyl ether, producing MA-PLA₄-PEG₁₄-PLA₄-MA liquid polymerizable macromer. The liquid polymerizable macromer was mixed with 4-methoxyphenol was at a final concentration of 100 ppm and stored for 12 hours at 60 degrees Celsius. No change was observed in the product due to the storage at elevated temperature.

1. A method of making a degradable, additive-blended polymeric material comprising:
(a) providing a first polymeric material for a central moiety (Aₙ),
(b) contacting the first polymeric material with a monomer B,
(c) initiating a reaction between the first polymeric material and the monomer by microwave radiation, thereby forming a block copolymer with blocks composed of the starting polymeric material and the polymer of the monomer (Bₘ-Aₙ-Bₘ),
(d) contacting the block copolymer with a second monomer (R),
(e) initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(f) contacting one or more liquid, polymerizable macromer(s) with initiator(s),
(g) blending the liquid, polymerizable mixture with additive(s), and
(h) exposing the additive-blended, liquid polymerizable macromer(s) to as external stimulus to create free radicals for a period of time, thereby forming a degradable, additive-blended gel.

2. The method according to claim 1, wherein the external stimulus is microwave radiation.

3. The method according to claim 1, wherein the central moiety is a polyethylene glycol.

4. The method according to claim 1, wherein the central moiety is a polyethylene glycol with an average molecular weight of 200, 400 or 600.

5. The method of claim 1, where the reactions are performed until step g for molecules with a central moiety of polyethylene glycol with different average molecular weight, then these liquid, polymerizable macromers are mixed together before step h.

6. The method of claim 1, wherein at least one additive is a therapeutic agent.

7. The method of claim 1, wherein at least one additive is an analgesic.

8. The method of claim 1, wherein at least one additive is an antibiotic.

9. The method of claim 1, wherein at least one additive is an anti-inflammatory drug.

10. The method of claim 1, wherein the external stimulus for polymerization is UV irradiation.

11. The method of claim 1, wherein the external stimulus for polymerization is heating.

12. The method of claim 1, wherein the external stimulus for polymerization is visible light.

13. The method of claim 1, wherein the additive-blended liquid polymerizable mixture of step (g) is applied to a medical device surface before step (h).

14. The method of claim 1, wherein the additive-blended polymerizable mixture of step (g) is applied on a porous metal surface before step (h).

15. The method of claim 1, wherein the block copolymer is washed with a solvent or solvents between steps (c) and (d).

16. The method of claim 1, wherein the liquid polymerizable macromer is washed with a solvent or solvents between steps (e) and (f).

17. A degradable, additive-blended polymeric material made by a method comprising the steps of:
(a) providing a first polymeric material for a central moiety (Aₙ),
(b) contacting the first polymeric material with a monomer B,
(c) initiating a reaction between the first polymeric material and the monomer by microwave radiation, thereby forming a block copolymer with blocks composed of the starting polymeric material and the polymer of the monomer (Bₘ-Aₙ-Bₘ),
(d) contacting the block copolymer with a second monomer (R),
(e) initiating a reaction between the chain ends of the block copolymer and the second monomer using microwave radiation, thereby forming a liquid polymerizable macromer composed of a cross-linkable moiety ((Bₘ-Aₙ-Bₘ—R) or (R—Bₘ-Aₙ-Bₘ—R)),
(f) contacting one or more liquid, polymerizable macromer(s) with initiator(s),
(g) blending the liquid, polymerizable mixture with additive(s), and
(h) exposing the additive-blended, liquid polymerizable macromer(s) to microwave radiation to create free radicals for a period of time, thereby forming a degradable, additive-blended gel.

18. The degradable, additive-blended polymeric material according to claim 17, wherein the central moiety is a polyethylene glycol.

19. The degradable, additive-blended polymeric material according to claim 17, wherein at least one additive is a therapeutic agent.

20. The degradable, additive-blended polymeric material according to claim 19, wherein the therapeutic agent is selected from the group consisting of an analgesic, and anti-inflammatory drug and an antibiotic.
